(19) United States
(12) Patent Application Publication
(10) Pub. No.: US 2023/0324108 A1
(43) Pub. Date: Oct. 12, 2023

(54) REFRIGERATOR AND METHOD OF CONTROLLING THE SAME

(71) Applicant: SAMSUNG ELECTRONICS CO., LTD., Suwon-si (KR)

(72) Inventors: Daewhan KIM, Suwon-si (KR); Kookjeong SEO, Suwon-si (KR); Wonjae YOON, Suwon-si (KR)

(73) Assignee: SAMSUNG ELECTRONICS CO., LTD., Suwon-si (KR)

(21) Appl. No.: 18/209,602

(22) Filed: Jun. 14, 2023

Related U.S. Application Data
(63) Continuation of application No. PCT/KR2021/018634, filed on Dec. 9, 2021.

(30) Foreign Application Priority Data
Dec. 18, 2020 (KR) 10-2020-0178205

(51) Int. Cl. F25D 23/12; B67D 3/00; C02F 1/68

(52) U.S. Cl. CPC F25D 23/126; B67D 3/0009; B67D 3/0019; C02F 1/68; F25D 2323/121; B67D 2210/0001; B67D 2210/0012; B67D 2210/00036

(57) ABSTRACT

A refrigerator and a method of controlling the same, the refrigerator including: a main body including a storage compartment; a door to open and close the storage compartment; and a beverage provider arrangeable with respect to the main body or the door. The beverage provider including: a water supplying unit; a storage to store water supplied by the water supplying unit; a filter, arrangeable inside the storage, and accommodate beverage powder so that ingredients from the beverage powder accommodated in filter are leached into the water; a sensor to obtain information related to a concentration of a beverage produced to contain the ingredients of the beverage powder in the water; and a processor configured to identify the concentration of the beverage based on the obtained information, and control the water supplying unit to supply the water in an amount which is adjusted based on the identified concentration.

### CROSS-REFERENCE TO RELATED APPLICATION

This application is a continuation application, under 35 U.S.C. § 111(a), of international application No. PCT/KR2021/018634, filed on Dec. 9, 2021, which claims priority under 35 U. S. C. § 119 to Korean Patent Application No. 10-2020-0178205 filed on Dec. 18, 2020, the disclosures of which are incorporated by reference herein in their entireties.

### BACKGROUND

### Field

The disclosure relates to a refrigerator and a method of controlling the same, and more particularly to a refrigerator capable of providing a beverage and a method of controlling the same.

### Description of Related Art

A refrigerator refers to an apparatus that allows stored things to remain fresh for a long period of time by cold air supplied into a storage compartment. The cold air supplied into the storage compartment is generated by heat exchange of the refrigerant, and evenly distributed in the storage compartment by convection, thereby storing food at a desired temperature.

The refrigerator includes the storage compartment provided in a main body forming an outer appearance and opened frontwards to accommodate food therein. Further, a door is provided in the front of the storage compartment so that the storage compartment can be opened and closed. The door is hinged to the main body and rotated to open and close the storage compartment.

The refrigerator may include a storage, such as a water tank, mounted to the door or the like and storing cold water. The refrigerator is provided with a water supply device to fill the storage with purified cold water, so that a user can take out cold water when needed.

### SUMMARY

According to an embodiment of the disclosure, a refrigerator includes: a main body including a storage compartment; a door to open and close the storage compartment; and a beverage provider arrangeable with respect to the main body or the door, the beverage provider including: a water supplying unit; a storage to store water supplied by the water supplying unit; a filter, arrangeable inside the storage, to accommodate beverage powder so that while the filter is arranged inside the storage, ingredients from the beverage powder accommodated in filter are leached into the water stored in the storage; a sensor to obtain information related to a concentration of a beverage produced to contain the ingredients of the beverage powder in the water; and a processor configured to identify the concentration of the beverage based on the obtained information, and control the water supplying unit to supply the water in an amount which is adjusted based on the identified concentration.

The sensor may be among a sensor unit which includes a contact or non-contact concentration sensor to detect the concentration of the beverage based on electrical conductivity.

The sensor may be among a sensor unit which includes a weight sensor under the storage, and the processor may be configured to identify weight of the beverage powder accommodated in the filter based on a detection result of the weight sensor, and identify the concentration of the beverage based on the identified weight of the beverage powder and the amount of the water supplied to the storage.

The sensor may be among a sensor unit which includes a mounting sensor to detect whether the storage is attached or detected with respect to the beverage provider, and the processor may be configured to count a beverage preparation time from a point in time when the mounting sensor detects that the storage is attached or detected with respect to the beverage provider.

The processor may be configured to identify the concentration of the beverage based on a preparation time of the beverage exceeding a predetermined reference period of time.

The processor may be configured to control the water supplying unit to supply the cold water to the storage, based on the concentration of the beverage identified being higher than a concentration set by a user.

The processor may be configured to output a message informing that preparation of the beverage is complete, based on the concentration of the beverage identified to reach a concentration set by a user.

The processor may be configured to output a message that an additional leaching time is required, based on the concentration of the beverage identified being lower than a concentration set by a user.

The refrigerator may further include an input unit to receive an input of a user, and the processor may be configured to control the water supplying unit to supply the water in the amount which is adjusted based on a water supply amount or concentration of the beverage that is input through the input unit.

The refrigerator may further include an input unit to allow a selection to operate the beverage provider in either a water purification mode to provide purified water or a beverage mode to provide the beverage.

The processor may be configured to: identify a water level of the water in the storage through the sensor in the water purification mode, and control the water supplying unit to maintain the cold water in the storage at a predetermined water level based on a result of the water level identified.

The processor may be configured to: identify a water level of the water in the storage based on an input of a user to switch operation of the beverage provider from the water purification mode to the beverage mode, and perform control not to switch to the beverage mode based on the water level being identified as a predetermined level or higher.

The input unit may further receive an input of a user to select a water supply amount or concentration of a beverage in the beverage mode, and the processor may be configured to control the water supplying unit to supply the water in the amount which is adjusted based on the selected water supply amount or concentration, to the storage.

The beverage powder may include coffee power, and the beverage may include cold brew coffee.

The refrigerator may further include a communicator to communicate with a server, the beverage powder may include coffee powder, and the processor may be configured to identify a kind of the coffee powder which is fed into the filter, receive information about a water supply amount or leaching time corresponding to the kind of the coffee powder identified from the server, and control cold brew coffee to be prepared based on the received information.

The refrigerator further includes a communicator, and the processor may transmit and receive information about operations of the beverage provider to and from an external device through the communicator.

According to an embodiment of the disclosure, a method of controlling a refrigerator is provided. The refrigerator includes a beverage provider which is arrangeable with respect to a main body or a door and includes a water supplying unit and a storage to store water supplied by the water supplying unit. The method of controlling the refrigerator includes: performing filtering to leach ingredients from beverage powder, which is accommodated in a filter into cold water supplied to the storage, the filter being arrangeable inside the storage of the beverage provider; obtaining, by a sensor, information related to a concentration of a beverage produced to contain the ingredients of the beverage powder in the cold water; and identifying the concentration of the beverage based on the obtained information, and controlling an amount of the water supplied by the water supplying unit to be adjusted based on the identified concentration.

The method may further include identifying the concentration of the beverage when a preparation time of the beverage exceeds a predetermined reference period of time.

The method may further include controlling water to be supplied to the storage based on the concentration of the beverage identified being higher than the concentration set by a user.

The method may further include controlling a message that preparation of the beverage has is complete, to be output based on the concentration of the beverage identified to reach a concentration set by a user.

The method may further include controlling a message that an additional leaching time is required, to be output based on the concentration of the beverage identified being lower than a concentration set by a user.

### BRIEF DESCRIPTION OF THE DRAWINGS

FIG. 1 is a perspective view of a refrigerator according to an embodiment of the disclosure.

FIG. 2 is a front view of the refrigerator of FIG. 1 when a door is opened according to an embodiment of the disclosure.

FIG. 3 is a perspective view of a refrigerator with a beverage provider according to an embodiment of the disclosure.

FIG. 4 is a schematic view of a water supplying unit according to an embodiment of the disclosure.

FIG. 5 is a perspective view illustrating that a storage is attached to and detached from the beverage provider of FIG. 3 according to an embodiment of the disclosure.

FIG. 6 is a perspective view illustrating that a filter and a storage are coupled and separated in the beverage provider of FIG. 3 according to an embodiment of the disclosure.

FIGS. 7 and 8 are exploded perspective views illustrating a coupling relationship of a filter of FIG. 5 according to an embodiment of the disclosure.

FIG. 9 illustrates an example that a sensor is provided in a beverage provider of a refrigerator according to an embodiment of the disclosure.

FIG. 10 is a view for describing a principle of measuring the concentration of solution through a non-contact concentration sensor according to an embodiment of the disclosure.

FIG. 11 illustrates another example that a sensor is provided in a beverage provider of a refrigerator according to an embodiment of the disclosure.

FIG. 12 illustrates still another example that a sensor is provided in a beverage provider of a refrigerator according to an embodiment of the disclosure.

FIG. 13 is a control block diagram of a refrigerator according to an embodiment of the disclosure.

FIG. 14 illustrates an example that an option selector of a beverage provider is provided in a refrigerator according to an embodiment of the disclosure.

FIG. 15 is a view for describing operations according to selection of a water purification mode in FIG. 14 according to an embodiment of the disclosure.

FIG. 16 is a view for describing operations according to selection of a beverage mode in FIG. 14 according to an embodiment of the disclosure.

FIG. 17 illustrates an example that a message related to beverage preparation is output according to an embodiment of the disclosure.

### DETAILED DESCRIPTION

Below, embodiments according to the disclosure will be described in detail with reference to accompanying drawings. In the drawings, like numerals or symbols refer to like elements having substantially the same function, and the size of each element may be exaggerated for clarity and convenience of description. However, the configurations and functions illustrated in the following exemplary embodiments are not construed as limiting the disclosure and the key configurations and functions. In the following descriptions, details about publicly known functions or features will be omitted if it is identified that they cloud the gist of the disclosure.

In the following exemplary embodiments, terms ‘first’, ‘second’, etc. are only used to distinguish one element from another, and singular forms are intended to include plural forms unless otherwise mentioned contextually. In the following exemplary embodiments, it will be understood that terms ‘comprise’, ‘include’, ‘have’, etc. do not preclude the presence or addition of one or more other features, numbers, steps, operation, elements, components or combination thereof. In addition, a ‘module’ or a ‘portion’ may perform at least one function or operation, be achieved by hardware, software or combination of hardware and software, and be integrated into at least one module. In the disclosure, at least one among a plurality of elements refers to not only all the plurality of elements but also both each one of the plurality of elements excluding the other elements and a combination thereof.

An aspect of the disclosure is to provide a refrigerator, of which a water supply device is used to provide a beverage, and a method of controlling the same.

As described above, in a refrigerator according to the disclosure and a method of controlling the same, a water supply device provided in the refrigerator is used to provide a beverage such as cold brew coffee.

In addition, a user is allowed to select a water purification mode and a beverage mode as necessary, and select a water supply amount or a concentration in the beverage mode, thereby making a beverage suitable for his/her own tastes.

Further, it is more convenient for a user because a guide related to beverage preparation is provided.

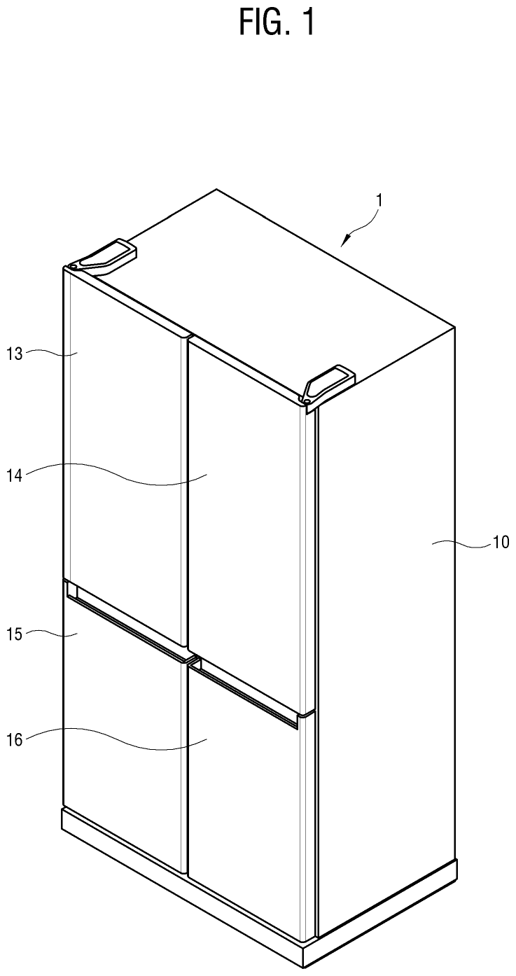

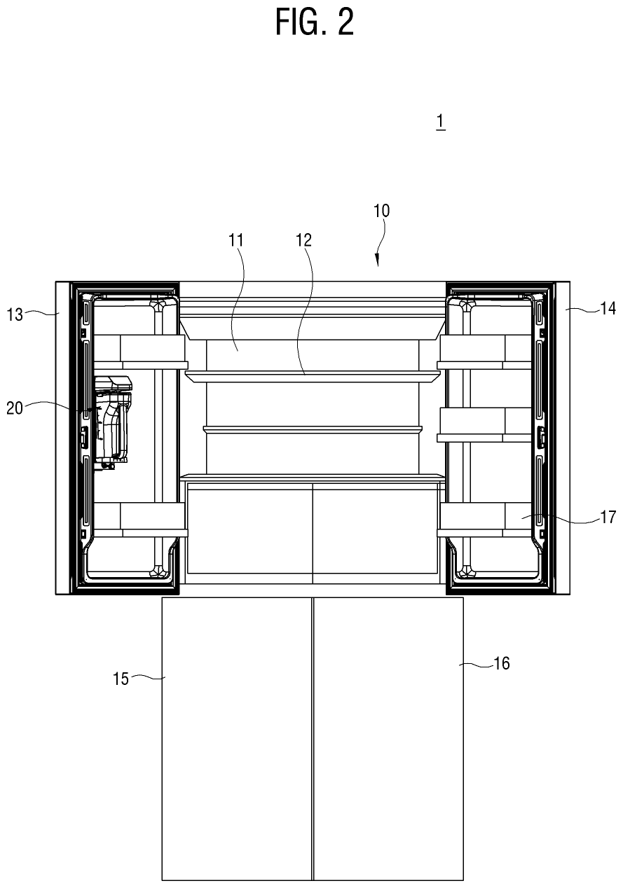

FIG. 1 is a perspective view of a refrigerator according to an embodiment of the disclosure, and FIG. 2 is a front view of the refrigerator of FIG. 1 when a door is opened.

According to an embodiment of the disclosure, a refrigerator 1 may, for example, be implemented by a general, double-, triple-, or quadruple-door refrigerator classified according to the number and opening types of doors. Further, the refrigerator 1 according to an embodiment of the disclosure may, for example, be implemented by a 1-EVA, 2-EVA, or 3-EVA refrigerator classified according to the number of evaporators for supplying cold air.

According to an embodiment of the disclosure, the refrigerator 1 may include an indirect- or direct-cooling type stand-up or built-in refrigerator. Further, the refrigerator 1 according to an embodiment may include a refrigerator with a refrigerator compartment and a freezer compartment for making ice, a freezer with only the freezer compartment, or an ice maker only for making ice.

As described above, the refrigerator 1 according to the disclosure is not limited to the structure or purpose described in the following embodiments, but may be implemented by any type of refrigerator having one or more refrigerator compartments or freezer compartments.

According to an embodiment, the refrigerator 1 includes a main body 10 forming an outer appearance as shown in FIGS. 1 and 2.

As shown in FIG. 2, the main body 10 is internally provided with a storage compartment 11 to store food and the like things. There may be provided a plurality of storage compartments 11. According to an embodiment, the storage compartments 11 may be partitioned by a partition wall.

According to an embodiment, the plurality of storage compartments 11 may include a freezer compartment for freezing stored things, and a refrigerator compartment for refrigerating stored things. The storage compartment 11 may be provided with a plurality of shelves 12 so that a user can put things stored to be refrigerated or frozen on the shelves 12.

The main body 10 is mounted with a cooling system for cooling the storage compartment 11 into a low temperature. According to an embodiment, the cooling system includes a compressor for compressing a refrigerant having low temperature and low pressure; a condenser for condensing the refrigerant, which has been compressed to have high temperature and high pressure by the compressor, by radiating heat to the outside; an expander for decompressing the refrigerant which has been condensed by the condenser; and an evaporator for evaporating the refrigerant, which has been expanded by the expander, by taking heat from air circulated in each storage compartment 11.

In the front of the main body 10, doors 13, 14, 15 and 16 are provided for opening and closing the storage compartments 11. The refrigerator 1 may include a plurality of doors 13, 14, 15 and 16, so that a user can open the doors 13, 14, 15 and 16 to store things in the storage compartment 11.

The doors 13, 14, 15 and 16 may include a plurality of baskets 17 to store things on an inner surface thereof, i.e., a surface facing the storage compartment 11 when the doors 13, 14, 15 and 16 are closed.

According to an embodiment of the disclosure, the refrigerator 1 includes a beverage provider 20 that provides a beverage to a user.

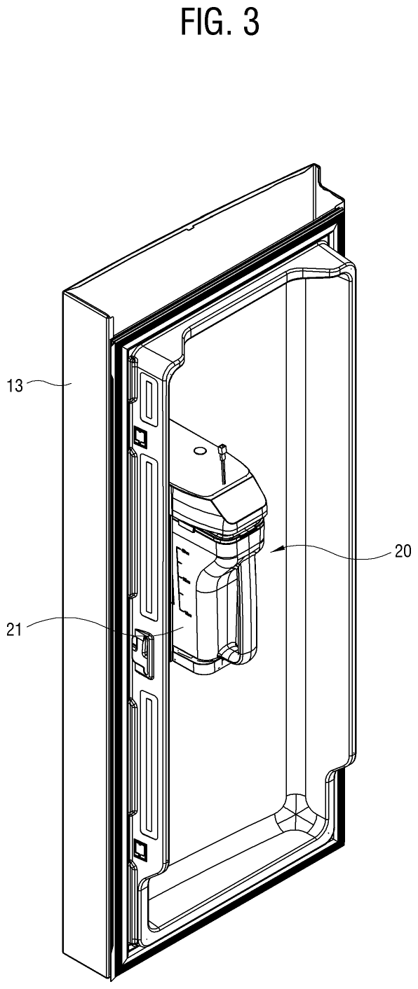

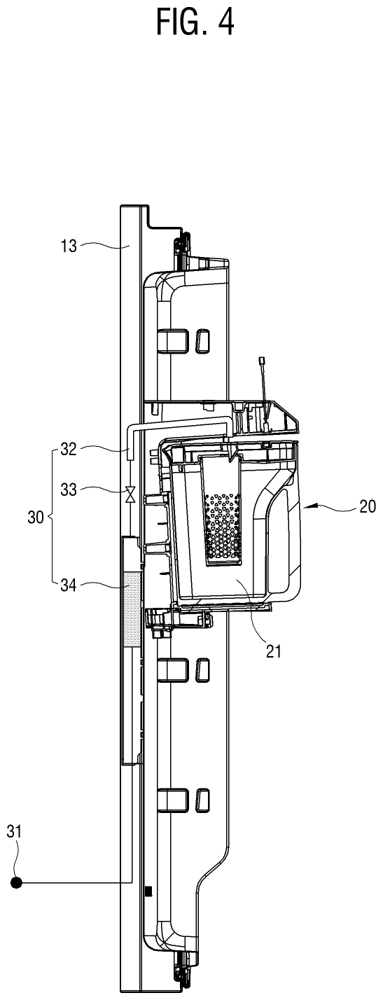

FIG. 3 is a perspective view of a refrigerator with a beverage provider according to an embodiment of the disclosure, and FIG. 4 is a schematic view of a water supplying unit.

The beverage provider 20 includes a water supplying unit 30 connected to a water supplying source 31 and supplying water, i.e., cold water; and a storage 21 storing the cold water supplied by the water supplying unit 30.

FIG. 3 shows a case where the beverage provider 20 is provided in any one door 13, but there are no limits to the installation position of the beverage provider 20 according to the disclosure. According to an embodiment of the disclosure, the beverage provider 20 may be provided in any one of the main body 10 or the doors 13, 14, 15 and 16 of the refrigerator 1.

As shown in FIG. 4, the water supplying unit 30 includes a water supplying pipe 32 providing a channel through which water is supplied from the water supplying source 31. The water supplying pipe 32 may be, but not limited thereto, implemented by a pipe made of a metal or resin material.

The water supplying pipe 32 may be provided with a flow valve 33 to adjust the amount or speed of water supplied. Further, the water supplying unit 30 may further include a purification filter 34 disposed on the water supplying pipe 32 and purifying water.

According to an embodiment, the water supplying pipe 32 may be installed inside the doors 13, 14, 15 and 16 or the main body 10 in the form of being bent a plurality of times, so that water can be cooled and then supplied to the storage 21. Therefore, water flowing through the water supplying pipe 32 is cooled and maintained cool by exchanging heat with the inside of the storage compartment 11, e.g., the refrigerator compartment or the freezer compartment while staying in the doors 13, 14, 15 and 16 or the main body 10, so that cold water can be supplied to the storage 21.

FIG. 4 shows an example that the water supplying unit 30 is provided in the door 13. According to the disclosure, there are no limits to the installation position of the water supplying unit 30. In other words, the elements 31, 32, and 33 of the water supplying unit 30 may be provided at various positions of the main body 10 or the doors 13, 14, 15 and 16 of the refrigerator 1, and may be distributively positioned. Further, the water supplying unit 30 according to the disclosure may be not included in the beverage provider 20 but provided as a separate element from the refrigerator 1.

The storage 21 may include a storage tank (hereinafter referred to as a pitcher or a kettle) provided as a container in which cold water supplied through the water supplying unit 30 is stored.

According to an embodiment, the storage 21 may be detachably installed in a supporter 22 provided in any one of the storage compartments 11, e.g., the doors 13, 14, 15 and 16 or the main body 10.

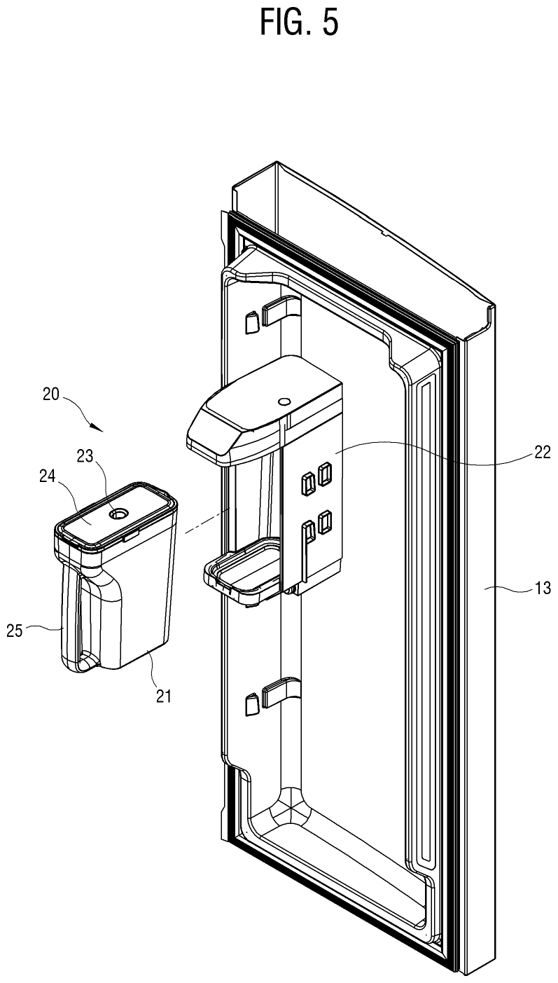

FIG. 5 is a perspective view illustrating that a storage is attached to and detached from the beverage provider of FIG. 3.

Any one of the main body 10 or the door 13, 14, 15 or 16 of the refrigerator 1 may include a supporter 22 to which the storage 21, i.e., the storage tank is coupled or mounted. The supporter 22 may, for example, be installed in the form of being coupled or supported onto the inner surface of the door 13 provided with the beverage provider 20.

The supporter 22 supports the storage 21 mounted thereto not to move when the door 13 is opened and closed. To this end, the supporter 22 may have a structure corresponding to the shape of the storage 21.

However, the detachable structure of the storage 21 according to the disclosure is not limited to that shown in FIG. 5. For example, according to an alternative embodiment, the storage 21 may be directly coupled and supported to the backset or the like of the door 13 without any separate supporter.

The storage 21 may include a water supplying hole 23 in an upper portion thereof, through which cold water flows in. The water supplying hole 23 is connected to the water supplying pipe 32 of the water supplying unit 30, so that the storage 21 can be filled with cold water supplied through the water supplying unit 30. The beverage provider 20 further includes a top cover 24 detachably mounted to an upper portion of the storage 21, and the water supplying hole 23 may, for example, be provided in the top cover 24.

The storage 21 may have various shapes forming a space to be filled with cold water or the like liquid. For example, the storage 21 may be shaped like, but not limited to, a hexahedron.

The storage 21 includes a handle 25 at one side thereof, so that a user can easily detach the storage 21 from the supporter 22 and drink a beverage of the storage 21.

In the refrigerator 1 according to an embodiment of the disclosure, the beverage provider 20 further include a filter 40 to accommodate beverage powder therein.

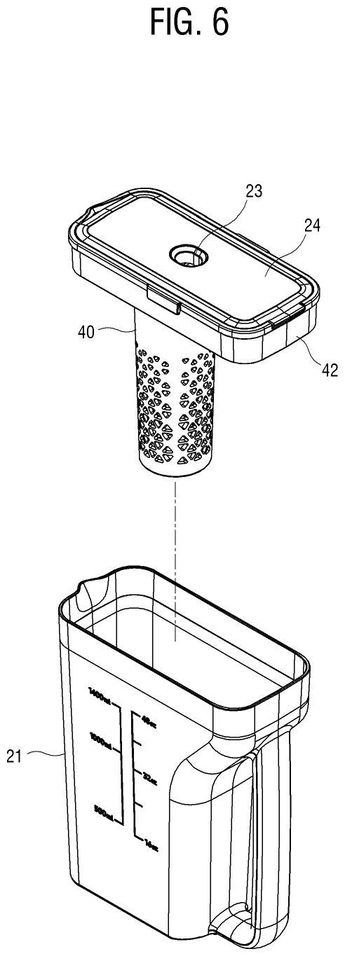

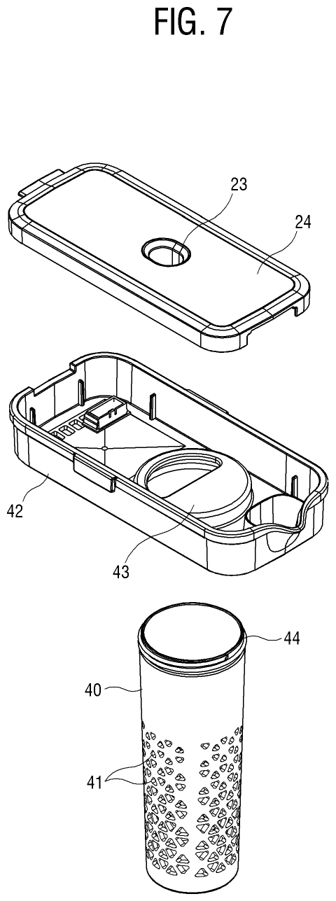

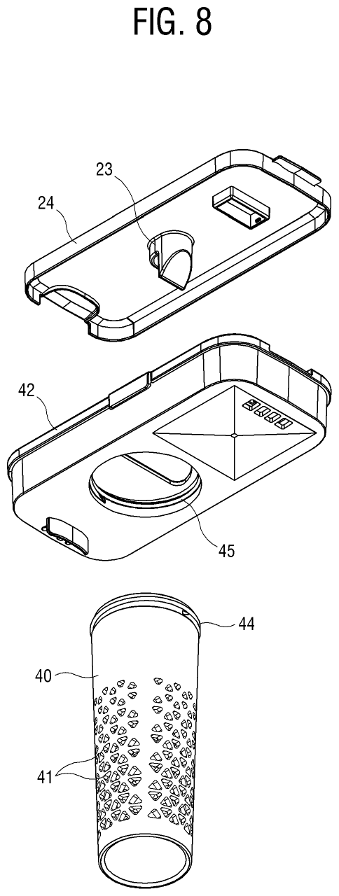

FIG. 6 is a perspective view illustrating that a filter and a storage are coupled and separated in the beverage provider of FIG. 3, and FIGS. 7 and 8 are exploded perspective views illustrating a coupling relationship of a filter of FIG. 5.

As shown in FIG. 6, the storage 21 is internally provided with the filter 40. The filter 40 may be configured to be coupled to and separated from the storage 21.

According to an embodiment, as shown in FIGS. 6 to 8, the beverage provider 20 may further include a filter mounting unit 42 coupled to the top cover 24, and the filter 40 may be coupled or mounted to the filter mounting unit 42.

As shown in FIGS. 7 and 8, the filter 40 may, for example, be provided as a hollow shape, such as a cylinder, forming a space to accommodate the beverage powder therein. According to the disclosure, there are no limits to the shape of the filter 40, and the filter 40 may have various shapes as long as it can accommodate the beverage powder therein.

The filter 40 is formed with a plurality of through holes 41 on the outer surface thereof, so that water can flow into the filter 40 through the through hole 41.

Specifically, when the filter 40 is mounted to the filter mounting unit 42 while accommodating the beverage powder therein and the filter mounting unit 42 is coupled to the storage 21, water, i.e., cold water supplied to the storage 21 flows into the filter 40 the through hole 41. Thus, the filter 40 performs filtering the beverage powder accommodated therein so that ingredients can leach from the beverage powder into cold water. By the filtering, the beverage powder itself is filtered by the filter 40, and the ingredients of the beverage powder are leached into cold water outside the filter 40. Therefore, as time elapses, the cold water in the storage 21 contains the ingredients of the beverage powder, thereby preparing a beverage.

According to an embodiment, the filter 40 may be coupled to a filter cover 43 provided in the filter mounting unit 42. As shown in FIGS. 7 and 8, the filter 40 may, for example, be formed with a screw thread 44 along the outer circumference in an upper portion thereof, and coupled to a corresponding inner circumference 45 of the filter cover 43 by rotation. However, the coupling method of the filter 40 is not limited to the rotation, but may alternatively include a fitting coupling method based on an elastic body.

As shown in FIGS. 6 to 8, the storage 21, the filter 40, the filter mounting unit 42 and the top cover 24 of the beverage provider 20 are implemented in the form of being coupled to and separated from one another, so that a user can separate and wash them as necessary.

There are no limits to the kinds of beverage powder accommodated in the filter 40, and the beverage powder includes coffee, tea, and the like powder of various raw materials, which can be leached into water or the like liquid to prepare a beverage.

In the refrigerator 1 according to an embodiment of the disclosure, the beverage provider 20 may be implemented as a cold brew coffee machine that prepares cold brew coffee by leaching coffee powder accommodated in the filter 40 into cold water supplied to the storage 21.

Alternatively, in the refrigerator 1 according to an embodiment, the beverage provider 20 may operate as a water purifier that purifies and provides water supplied from the water supplying source 31. In other words, when the filter 40 is removed from the beverage provider 20, cold water purified by the purification filter 34 of the water supplying unit 30 is supplied to and retained in the storage 21. Therefore, the beverage provider 20 functions as the water purifier that provides purified water.

The beverage provider 20 may include at least one sensor.

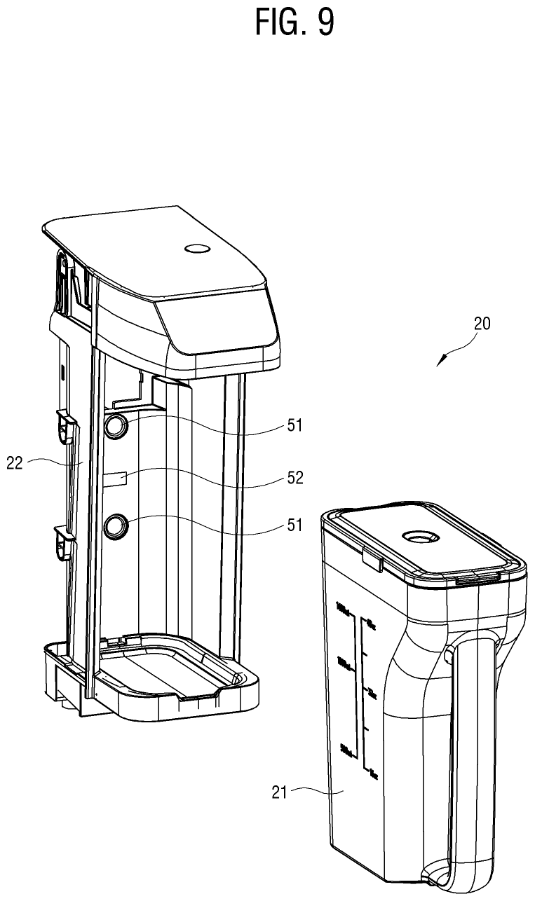

FIG. 9 illustrates an example that a sensor is provided in a beverage provider of a refrigerator according to an embodiment of the disclosure.

According to an embodiment, the beverage provider 20 includes a mounting sensor 51 that detects whether the storage 21 for storing a beverage is mounted to or detached from the supporter 22. As shown in FIG. 9, the mounting sensor 51 may for example include a pressure sensor or an infrared sensor installed in the supporter 22.

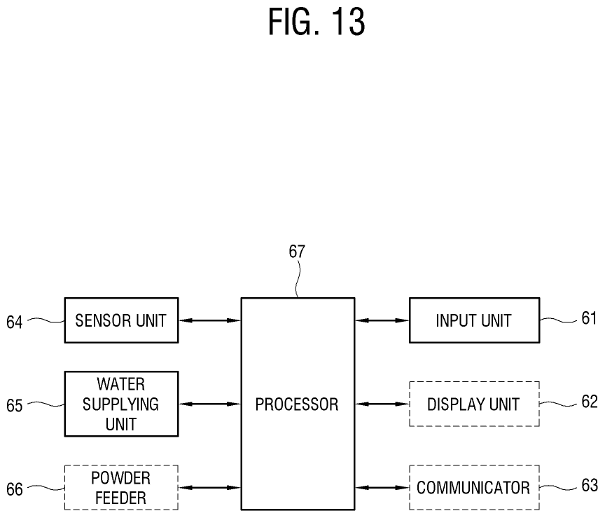

The mounting sensor 51 may detect whether the storage 21 is mounted to the supporter 22 provided in any one of the main body 10 or the doors 13, 14, 15 and 16 of the refrigerator 1, and output a detection result to a processor 67 (see FIG. 13). The processor 67 may detect that the storage 21 is mounted to the supporter 22 through the mounting sensor 51, and count a beverage preparation time from the mounting detection time.

In the refrigerator 1 according to an embodiment of the disclosure, the sensor provided in the beverage provider 20 includes a sensor capable of obtaining information about the concentration of a beverage in the storage 21, i.e., a storage tank. The beverage provider 20 may for example include a non-contact type or contact concentration sensor to obtain information about the concentration of a beverage.

According to an embodiment, the beverage provider 20 may include a non-contact concentration sensor 52. As shown in FIG. 9. the non-contact concentration sensor 52 may, for example, be installed in the supporter 22 of the beverage provider 20.

The non-contact concentration sensor 52 may for example include an optical sensor.

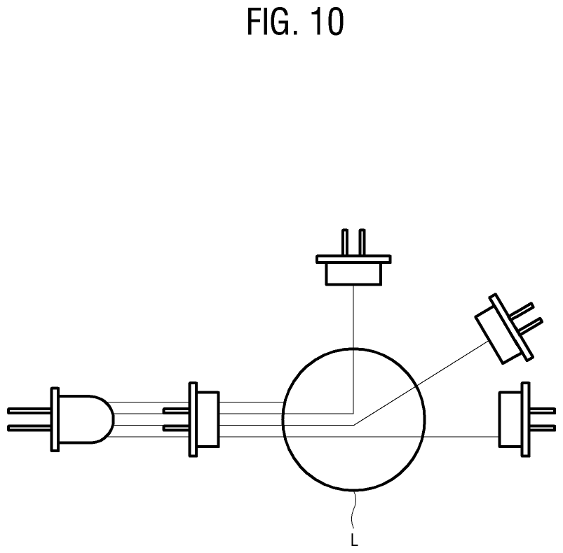

FIG. 10 is a view for describing a principle of measuring the concentration of solution through a non-contact concentration sensor.

When cold water is supplied to the storage 21 in the state that the beverage powder is accommodated in the filter 40, the ingredients of the beverage powder accommodated in the filter 40 are leached into the cold water, thereby filling the storage 21 with a prepared beverage.

As shown in FIG. 10, the non-contact concentration sensor 52 may emit light to a measurement target solution L, i.e., the beverage filled in the storage 21, measure the electrical conductivity (EC) of the beverage, and obtain the information about the concentration of the beverage based on a relationship between the electrical conductivity and the concentration.

The concentration of water may be measured based on the electrical conductivity (EC), and the unit of electrical conductivity (EC) is ‘dS/m’ or ‘mmho/cm’ based on a relationship of the following equation 1.

1dS/m=1 mmho/cm [the equation 1]

The electrical conductivity (EC) measured by the non-contact concentration sensor 52 may be converted into total dissolved solids (TDS) based on a relationship of the following equation 2.

TDS(mg/L or ppm)=EC(dS/m)×640(EC from 0.1 to 5 dS/m)

TDS(mg/L or ppm)=EC(dS/m)×800(EC>5 dS/m) [the equation 2]

The non-contact concentration sensor 52 may obtain the electrical conductivity as information about the concentration of the beverage filled in the storage 21 by the same method as above, and output the obtained information, i.e., the electrical conductivity to the processor 67. The processor 67 converts the electrical conductivity obtained by the equation 2 into the concentration, thereby identifying the concentration of the beverage.

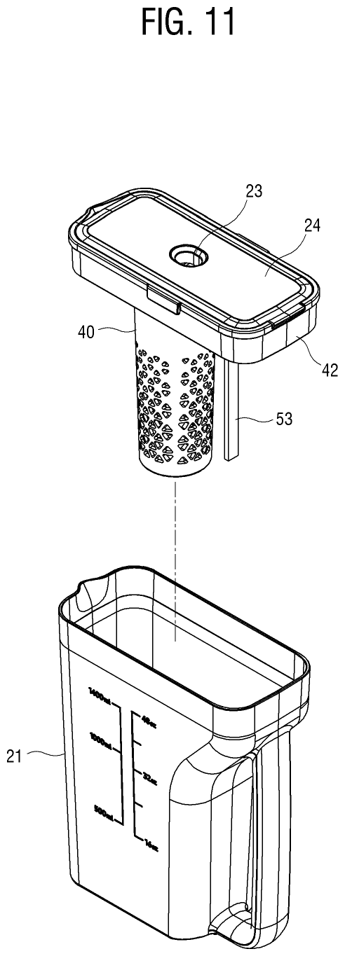

FIG. 11 illustrates another example that a sensor is provided in a beverage provider of a refrigerator according to an embodiment of the disclosure.

According to an embodiment, the beverage provider 20 may include a contact concentration sensor 53. As shown in FIG. 11, the contact concentration sensor 53 may, for example, be installed in the filter mounting unit 42 mountable to the upper portion of the storage 21. When the filter mounting unit 42 provided with the contact concentration sensor 53 as shown in FIG. 11 is coupled to the storage 21, the contact concentration sensor 53 comes into direct contact with the beverage filled in the storage 21.

The contact concentration sensor 53 may for example include an electrode-type conductivity sensor.

The contact concentration sensor 53 is in contact with the beverage of the storage 21 to measure the electrical conductivity (EC) of the beverage, thereby obtaining the information about the concentration of the beverage. The contact concentration sensor 53 may for example measure the electrical conductivity based on the quantity of electricity flowing between two sensitive portions positioned at the terminals of the sensor.

The electrical conductivity (EC) measured by the contact concentration sensor 53 may be converted in to the concentration (TDS) based on a relationship between the concentration and the electrical conductivity of the equation 2 as described above in relation to the non-contact concentration sensor 52.

The contact concentration sensor 53 may obtain the electrical conductivity as information about the concentration of the beverage filled in the storage 21 by the same method as above, and output the obtained information, i.e., the electrical conductivity to the processor 67. The processor 67 converts the electrical conductivity obtained by the equation 2 into the concentration, thereby identifying the concentration of the beverage.

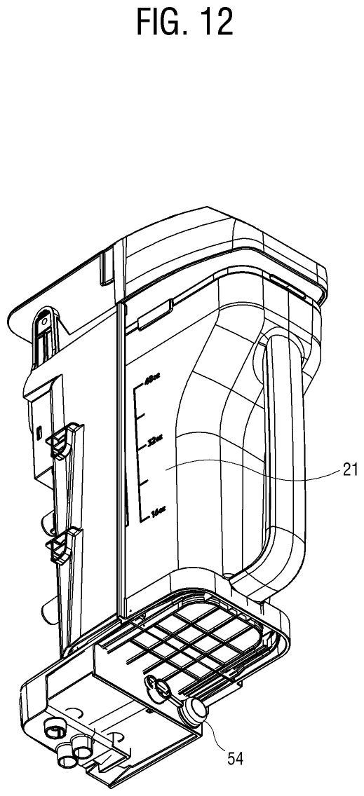

FIG. 12 illustrates still another example that a sensor is provided in a beverage provider of a refrigerator according to an embodiment of the disclosure.

According to an embodiment, the beverage provider 20 may include a weight sensor 54, i.e., a sensor for detecting the weight. As shown in FIG. 12, the weight sensor 54 is installed under the storage 21, i.e., a storage tank to be filled with the beverage.

The weight sensor 54 may for example include a load cell or a strain gauge that generates an electrical output in proportional to the magnitude of a load applied thereto.

According to an embodiment, the weight sensor 54 measures the weight of the storage 21 filled with the beverage prepared by the leaching from the beverage powder into cold water, thereby obtain information about the concentration of the beverage.

The weight sensor 54 may for example measure the total weight of the storage 21 filled with the beverage prepared by the leaching from the beverage powder.

The weight of the beverage powder may be obtained by subtracting the weight of the cold water supplied to the storage 21 and the weight of the storage 21 itself (i.e., the weight of the empty storage tank) from the measured total weight of the storage 21. Here, the weight of the cold water supplied to the storage 21 may be identified based on a preset amount of water supplied.

The weight sensor 54 may obtain the weight information as information about the concentration of the beverage filled in the storage 21 by the same method as above, and output the obtained weight information to the processor 67.

According to an embodiment, the weight sensor 54 may transmit the information about the total weight of the storage 21 filled with the beverage to the processor 67, so that the processor 67 can derive the weight of the beverage powder from the received information. Alternatively, the weight sensor 54 may derive the information about the weight of the beverage powder from the information about the total weight of the storage 21, and transmit the derived information to the processor 67.

The processor 67 may identify the concentration of the beverage based on the derived weight of beverage powder and the amount of cold water supplied to the storage 21 (the water supply amount).

According to an embodiment, the weight sensor 54 may be used as a water level sensor that detects the level of water (cold water) supplied to the storage 21. In other words, the level of water in the storage 21 is identified based on a detection result of the weight sensor 54, and the amount of water supplied to the storage 21 by the water supplying unit 30, i.e., the water supply amount is controlled according to the identified level of water.

According to an alternative embodiment, the beverage provider 20 may include a separate water level sensor, and the water level sensor may for example include a pressure sensor or an infrared sensor. The pressure sensor may for example installed under the storage 21, and the weight of the beverage powder may be identified based on a detection result of the pressure sensor.

In the refrigerator 1 according to an embodiment of the disclosure, the beverage provider 20 may obtain information about the concentration of the beverage from the sensor by one of the foregoing methods, and identify the concentration of the beverage based on the obtained information.

According to an embodiment, the beverage provider 20 may control the water supplying unit 30 to adjust the amount of cold water supplied to the storage 21 based on the concentration of the beverage identified as above. For example, the amount of water supplied to the storage 21 may be controlled by the flow valve 33 of the water supplying unit 30.

Meanwhile, according to an embodiment, the beverage provider 20 may further include a powder feeder to supply the beverage powder to the filter 40. The powder feeder may, for example, be implemented in the form of opening and closing the filter cover 43 or a separate feeding cover so that the beverage powder accommodated in a separate container can be automatically fed to the filter 40 by a predetermined amount.

According to an embodiment of the disclosure, the refrigerator 1 includes the beverage provider 20 as described above to selectively provide purified water (cold water) or a beverage prepared by leaching the ingredients from the beverage powder into cold water.

Below, it will be described that the beverage provider 20 of the refrigerator 1 according to an embodiment provides water or a beverage.

FIG. 13 is a control block diagram of a refrigerator according to an embodiment of the disclosure.

However, FIG. 13 merely shows an exemplary configuration of the refrigerator 1 according to an embodiment of the disclosure, and a refrigerator according to an alternative embodiment may be implemented by another configuration different from that shown in FIG. 13. In other words, the refrigerator 1 of the disclosure may include another element in addition to the elements shown in FIG. 13, or may exclude at least one element from the elements shown in FIG. 13. Further, the refrigerator 1 of the disclosure may be implemented by changing some elements in the configuration shown in FIG. 13.

The refrigerator 1 according to an embodiment of the disclosure may, as shown in FIG. 13, include an input unit 61.

The input unit 61 may receive a user's input. The input unit 61 transmits various preset control commands or unrestricted information to the processor 67 in response to a user's input.

The input unit 61 includes various input means for receiving a user's input. For example, the input unit 61 may include one or more buttons or input keys provided in the main body 10 or the door 13, 14, 15 or 16 of the refrigerator 1.

According to an embodiment, the input unit 61 may include an option selector 70 (see FIG. 13) provided as a user interface (UI) that gives a user an option to select the purified water or the beverage from the beverage provider 20.

The refrigerator 1 may include a display unit 62.

The display unit 62 may be configured to display various pieces of information related to the operations or functions of the refrigerator 1.

According to an embodiment, the display unit 62 may, for example, be installed in the front of any one of the doors 13, 14, 15 and 16, and include a display capable of displaying information thereon. Here, there are no limits to the types of the display. For example, the display may be implemented by various types such as a liquid crystal display (LCD), a light-emitting diode (LED), an organic light-emitting diode (OLED), etc.

The display may display a user interface (UI, hereinafter also referred to as graphic user interface (GUI)) for controlling various operations of the refrigerator 1 thereon. However, as necessary, the refrigerator 1 may not include the display but be implemented to display information through a display of a smartphone or the like external terminal device connected through a communicator 63 (to be described later).

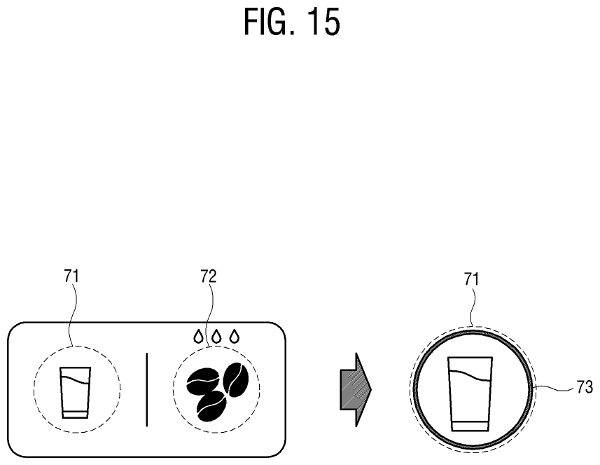

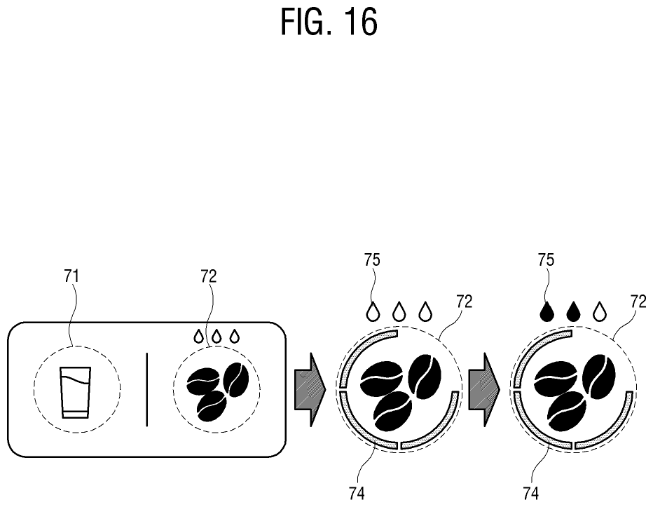

According to an embodiment, the display 62 may include a light emitting unit 73 (see FIG. 15) or 74 (see FIG. 16) showing the operating state of the refrigerator 1. The light emitting units 73 and 74 may, for example, be implemented by an LED and the like light emitting device that operates corresponding to a selected option of the beverage provider 20.

The refrigerator 1 may include the communicator 63.

The communicator 63 allows the refrigerator 1 to communicate with various external devices.

According to an embodiment, the communicator 63 may include a wireless interface. The wireless interface may for example employ wireless communication methods such as Wi-Fi, Bluetooth, ultrawideband (UWB), Zigbee, near field communication (NFC), and radio frequency (RF).

The wireless interface may be implemented by a communication circuitry including wireless communication modules (e.g., an S/W module, a chip, etc.) corresponding to various types of communication protocols.

According to an embodiment, the communicator 63 may include a wired interface. The wired interface may, for example, be implemented by a communication circuitry including a communication module capable of transceiving a signal/data based on standards such as Ethernet and universal serial bus (USB).

The external device communicating with the communicator 63 may for example include a smartphone, a tablet computer, a smart pad or the like terminal device. The terminal device may have an application for controlling the operations or functions of the refrigerator 1, for example, an application based on an Internet of things (IoT). A user may use the application installed in the terminal device to perform various settings related to the operations of the beverage provider 20 and check various messages based on data received from the refrigerator 1.

The external device communicating with the communicator 63 may for example additionally include a server. The server may include a database stored with various pieces of data related to preparation of a beverage, and thus a guide for preparing the beverage may be provided to the display 62 or the terminal device.

The refrigerator 1 may include a sensor unit 64.

The sensor unit 64 includes at least one sensor.

According to an embodiment, the sensor unit 64 may include the mounting sensor 51 to detect whether the storage 21 is attached or detached.

According to an embodiment, the sensor unit 64 includes at least one sensor capable of obtaining information about the concentration of the beverage in the storage 21. In other words, the sensor unit 64 may include at least one among the non-contact concentration sensor 52, the contact concentration sensor 53, and the weight sensor 54.

The operations of the sensors 51, 52, 53, and 54 included in the sensor unit 64 are the same as those of the embodiments described with reference to FIGS. 9 to 12.

According to an embodiment, the sensors 51, 52, 53, and 54 of the sensor unit 64 may operate periodically or when a predetermined event occurs, thereby outputting detection results to the processor 67.

The refrigerator 1 may further include a water supplying unit 65.

The water supplying unit 65 is equivalent to the water supplying unit 30 shown in FIG. 4, and may be installed in the main body 10 or the doors 13, 14, 15 and 16 of the refrigerator 1. In other words, the descriptions about the water supplying unit 30 according to the embodiment of FIG. 4 is equally applied to the water supplying unit 65 of FIG. 13, and thus repetitive descriptions will be avoided.

The refrigerator 1 may include a powder feeder 66.

The powder feeder 66 may for example include a container in which the beverage powder is accommodated, and a feeding device by which the beverage powder is automatically fed from the container to the filter 40. The powder feeder 66 may, for example, be implemented in the form of controlling the filter cover 43 or a separate feeding cover to be opened and closed so that the beverage powder accommodated in the container can be automatically fed to the filter 40 by a predetermined amount.

The refrigerator 1 may include the processor 67.

The processor 67 performs control for operating general elements of the refrigerator 1. The processor 67 may include a control program (or instruction) which performs such control operations, a nonvolatile memory in which the control program is installed, a volatile memory into which at least a part of the installed control program is loaded, and at least one processor, such as a microprocessor, an application processor, or a central processing unit (CPU), which executes the loaded control program.

The processor 67 performs computations, instructions, etc. for general operations performed by the refrigerator 1, for example, operations of the beverage provider 20, according to the control program.

According to an embodiment, the processor 67 may be implemented in the form of being included in the main SoC mounted to a printed circuit board (PCB) embedded in the main body 10 or the door 13, 14, 15 or 16 of the refrigerator 1.

The control program may include a program(s) achieved by at least one of a basic input/output system (BIOS), a device driver, an OS, a firmware, a platform, or an application. According to an embodiment, the application may be previously installed or stored when the refrigerator 1 is manufactured, or may be installed in the refrigerator 1 based on data of the application received from the outside when it is used in the future. The data of the application may, for example, be downloaded from an external server such as an application market to the refrigerator 1. Such an external server is merely an example of a computer program product according to the disclosure, but not limited thereto.

The control program may be recorded in a storage medium readable by a machine such as a computer. The machine-readable storage medium may be provided in the form of a non-transitory storage medium or non-volatile storage medium. Here, the term ‘non-transitory storage medium’ means a tangible device and does not include a signal (e.g., an electromagnetic wave), and this term does not distinguish between a case where data is semi-permanently stored in the storage medium and a case where data is temporarily stored. For example, the ‘non-transitory storage medium’ may include a buffer in which data is temporarily stored.

According to an embodiment, the processor 67 controls the beverage provider 20 of the refrigerator 1 to operate in either the water purification mode for providing the purified water (cold water) or the beverage mode for providing the beverage prepared by leaching ingredients from the beverage powder into cold water. The processor 67 may control the beverage provider 20 to operate in the water purification mode or the beverage mode in response to an option selected by a user through the input unit 61.

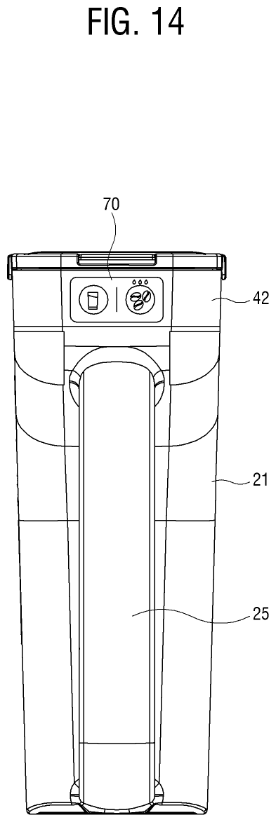

FIG. 14 illustrates an example that an option selector of a beverage provider is provided in a refrigerator according to an embodiment of the disclosure, FIG. 15 is a view for describing operations according to selection of a water purification mode in FIG. 14, and FIG. 16 is a view for describing operations according to selection of a beverage mode in FIG. 14.

As shown in FIG. 14, the refrigerator 1 according to an embodiment of the disclosure may include an option selector 70 as the input unit 61 in the filter mounting unit 42 coupled to the storage 21. The option selector 70 may, for example, be provided at one lateral side of the storage 21, where the handle 25 is positioned, so that a user can easily manipulate the option selector 70 in the state that the storage 21 is coupled to the supporter 22 of the door 13.

The option selector 70 may include a plurality of buttons 71 and 72 for selecting the option, i.e., the operation mode of the beverage provider 20. There are no limits to the types of buttons 71 and 72. For example, the buttons 71 and 72 may be formed elastically, and switches may be placed underneath the buttons 71 and 72 and operate in response to the manipulation of the buttons 71 and 72.

According to an embodiment, as shown in FIGS. 15 and 16, light emitting units 73 and 74 including the light emitting devices such as the LEDs may be provided around the buttons 71 and 72. The light emitting units 73 and 74 may operate corresponding to a user's manipulation, i.e., selection of the option for the corresponding buttons 71 and 72.

According to an embodiment, the option selector 70 may be implemented to include light emitting buttons in the form of a combination of the buttons 71 and 72 and the light emitting units 73 and 74 operating in response to the manipulation of the buttons 71 and 72.

However, according to the disclosure, the position and type of the option selector 70 are not limited to those shown in FIG. 14. For example, the option selector may be implemented as a UI including menu items, which is displayed on the display provided in any one of the doors 13, 14, 15 and 16 of the refrigerator 1.

A user may manipulate the first button 71 of the option selector 70 so that the beverage provider 20 can operate in the water purification mode. Here, a user may manipulate the first button 71 corresponding to the water purification mode in the state that the filter 40 is separated from the filter mounting unit 42.

When a user makes an input for the first button 71 corresponding to the water purification mode, the corresponding first light emitting unit 73 is turned on as shown in FIG. 15, thereby allowing a user to recognize that the water purification mode is selected.

According to an embodiment, the processor 67 may identify that a user's input of selecting the water purification mode is made when the first button 71 is pressed for a predetermined period of time, for example, 3 or more seconds.

The processor 67 controls the water supplying unit 65 to supply cold water to the storage 21 based on a user's input (manipulation) for the first button 71.

According to an embodiment, the processor 67 may control the water supplying unit 65 to fill the storage 21 with cold water up to a predetermined level corresponding to the water purification mode.

According to an embodiment, the water purification mode may also be referred to as an autofill mode.

In the refrigerator 1 according to an embodiment of the disclosure, the beverage provider 20 in the water purification mode, i.e., the autofill mode may periodically identify a water level of the storage 21 through the sensor unit 64, and, when it is identified that the water level is low, operate to automatically fill the storage 21 with cold water until the water level reaches a predetermined level, e.g., a full water level. In other words, the cold water of the storage 21 in the autofill mode may be controlled to always maintain a predetermined water level.

A user may manipulate the second button 72 of the option selector 70 so as to select the beverage mode of the beverage provider 20.

According to an embodiment, the beverage mode includes a cold brew mode that provides cold brew coffee prepared by leaching from the coffee powder accommodated in the filter 40 into cold water of the storage 21

In the refrigerator 1 according to an embodiment of the disclosure, the beverage provider 20 may be implemented to operate in any one of the autofill mode and the cold brew mode. Below, it will be described that the beverage provider 20 provides cold brew coffee.

When a user makes an input for the second button 72 corresponding to the beverage mode, i.e., the cold brew mode, the corresponding second light emitting unit 75 is turned on, thereby allowing a user to recognize that the cold brew mode is selected.

According to an embodiment, the processor 67 may identify that a user's input of selecting the cold brew mode is made when the second button 72 is pressed for a predetermined period of time, for example, 3 or more seconds.

According to an embodiment, when the beverage provider 20 operates in the autofill mode, and a user makes an input of selecting the cold brew mode in the state that the storage 21 is filled with cold water at a predetermined level or higher, the processor 67 controls mode switching not to be performed. In other words, the processor 67 may identify the water level of the storage 21 based on a user's input of switching the water purification mode (i.e., the autofill mode) of the beverage provider 20 over to the beverage mode (i.e., the cold brew mode), and perform control not to switch over to the beverage mode based on the water level identified as a predetermined level or higher.

In this case, the processor 67 may output a guidance message for guidance to empty the storage 21 and switch the mode through the display 62 provided in the refrigerator 1 itself or a smartphone or the like external device connected through the communicator 63.

According to an embodiment, the option selector 70 may allow the water supply amount or concentration for a beverage, i.e., cold brew coffee to be selected.

For example, in the state that the cold brew mode is selected by manipulating the second button 72 for a long period of time (e.g., 3 or more seconds), a user may manipulate the second button 72 for a short period of time to adjust the amount of water supplied to the storage 21.

According to an embodiment, the water supply amount of water to be supplied may be adjusted according to a plurality of levels, for example, four levels, as shown in FIG. 16. FIG. 16 shows an example that a user manipulates the second button 72 three times briefly (e.g., less than one second) to select a third level of the water supply amount. The second light emitting unit 74 may for example include four light emitting devices surrounding the second button 72, thereby notifying that the third level is selected corresponding to the selected water supply amount.

Based on a user's input (manipulation) of selecting the cold brew mode, the processor 67 controls the water supplying unit 65 to supply cold water to the storage 21. Here, the processor 67 may control the water supplying unit 65 to fill the storage 21 with cold water corresponding to the water supply amount selected through the second button 72.

In the embodiment of FIG. 16, the water supply amount is selected by way of example to select the concentration or the like of coffee. However, the option selector 70 may be implemented to select the concentration or the like of coffee in various forms. For example, the concentration of the cold brew coffee may be selected from among light, regular, strong, etc.

According to an embodiment, the option selector 70 may further include a third light emitting unit 75 that indicates the current concentration of a beverage prepared in the beverage provider 20.

The processor 67 obtains information about the concentration of the beverage in the storage 21 through the sensor of the sensor unit 64, and notify the information through the third light emitting unit 75.

Specifically, when the storage 21 is filled with cold water in the state that the beverage powder, i.e., the coffee powder is accommodated in the filter 40, the ingredients of the beverage powder are leached from the beverage powder into the cold water as time elapses, thereby preparing the cold brew coffee.

The processor 67 may obtain the information about the concentration of a beverage, i.e., cold brew coffee from the sensor of the sensor unit 64, e.g., any one of the non-contact concentration sensor 52, the contact concentration sensor 53, and the weight sensor 54, and identify the concentration of coffee based on the obtained information.

According to an alternative embodiment, the processor 67 may estimate the concentration of coffee in consideration of the amount of coffee powder fed to the filter 40, the amount of water supplied to the storage 21, and the leaching time (brewing time or preparing time) of the coffee powder. Here, the leaching time may be counted from a point in time when the mounting sensor 51 detects that the storage 21 is mounted to the supporter 22.

The processor 67 controls the third light emitting unit 75 to indicate the concentration of coffee identified or estimated as above. In the example shown in FIG. 16, the third light emitting unit 75 indicates that the concentration of coffee reaches a second level as time elapses.

According to an embodiment, the processor 67 may control the water supplying unit 65 to adjust the amount of cold water supplied to the storage 21, based on the concentration of coffee identified or estimated as described above.

For example, a user may feed a predetermined amount of coffee powder to the filter 40, select the cold brew mode through the option selector 70, and select the water supply amount for the beverage, i.e., cold brew coffee.

The processor 67 may obtain information about the concentration of coffee through the sensor unit 64 at predetermined time intervals while the ingredients of the coffee powder is leached into the cold air. The processor 67 may identify the current concentration of a beverage, i.e., coffee in the storage 21 based on the obtained information, and compare the identified concentration with the concentration set by a user, i.e., the concentration corresponding to the water supply amount selected by a user.

Here, the processor 67 may identify the concentration of the beverage, i.e., coffee in the storage 21 when the leaching time has exceeded a predetermined reference period of time, e.g., 12 hours, and compare the identified concentration with the concentration corresponding to the water supply amount selected by a user. The reference time may be set based on time to be typically taken in brewing coffee in cold water (for example, 8 to 18 hours), and may be modified by a user.

When it is identified as a result of the comparison that the current concentration of coffee is higher than the concentration set by a user, the processor 67 may control the water supplying unit 30 to additionally supply cold water to the storage 21 and dilute the coffee.

On the other hand, when it is identified as a result of the comparison that the current concentration of coffee reaches the concentration set by a user, the processor 67 may control the display 62 or the communicator 63 to output a message (or completion message) for informing that the beverage, i.e., cold brew coffee has been completely prepared. The completion message may, for example, be displayed through the display of the terminal device communicating with the communicator 63 and having an application installed for the refrigerator 1. Further, the completion message may be output as a sound through a loudspeaker provided in the refrigerator 1 itself or a loudspeaker of the terminal device.

Further, when it is identified as a result of the comparison that the current concentration of coffee is lower than the concentration set by a user, the processor 67 may increase the leaching time (or brewing time) because the ingredients are not sufficiently leached from the coffee powder.

Here, the processor 67 may control the display 62 or the communicator 63 to output a message (or guidance message) for guiding that an additional leaching time is required. The guidance message may, for example, be displayed through the display of the terminal device that communicates with the communicator 63 and has the application installed for the refrigerator 1. Further, the guidance message may be output as a sound through the loudspeaker provided in the refrigerator 1 itself, and the loudspeaker of the terminal device. Further, the display 62, the communicator 63, the loudspeaker, etc. may be controlled to output a warning message when a user removes the storage 21 in the state that the current concentration does not reaches the set concentration.

The processor 67 may output various messages related to the preparation of the beverage, i.e., coffee, based on the concentration information obtained using the sensor unit 64, the leaching time (brewing time) of coffee, etc. in the cold brew mode as described above.

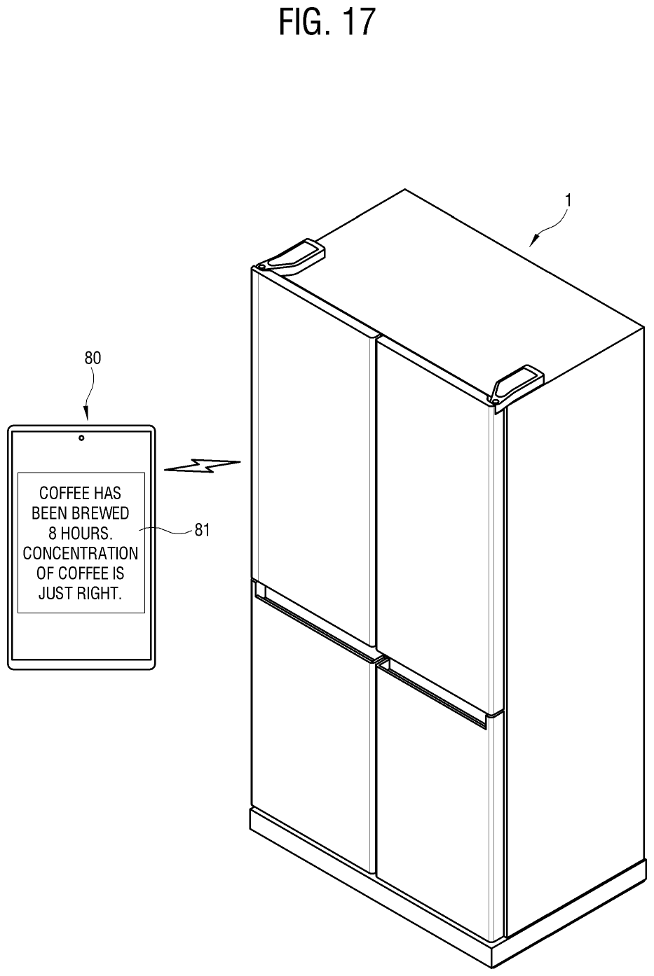

FIG. 17 illustrates an example that a message related to beverage preparation is output according to an embodiment of the disclosure.

The processor 67 may, as shown in FIG. 17, control the communicator 63 to output a message 81 that informs of the leaching time and current concentration of coffee through a terminal device 80. However, the method of outputting the message, the content of the message, etc. are not limited to those shown in FIG. 17. For example, the message may be output in various ways, such as using the display 62 or the loudspeaker provided in the refrigerator 1 itself. Further, the content of the message may be implemented in various ways. For example, the content of the message may be based on various pieces of information, such as guidance on the current setting option (the water supply amount, etc.), notification of time remaining to reach the concentration set by a user, or warning of a beverage left for a long period of time after preparation.

Meanwhile, the server and the like external device may include a database established storing information about an appropriate water supply amount, an appropriate leaching time, etc. corresponding to the kinds of coffee powder.

The processor 67 may receive data from the server through the communicator 63, and provide guidance on the preparation of the beverage, i.e., cold brew coffee to a user.

According to an embodiment, the kind of coffee powder fed into the filter 40 may be identified by the terminal device 80 having the application installed for the refrigerator 1. For example, the kinds of coffee powder may be identified by scanning a barcode, a quick response (QR) code, or the like image corresponding to the kind of coffee powder through the terminal device 80, or by an input made by a user in person.

When the kind of coffee powder is identified, the server may identify the water supply amount, etc. corresponding to the identified coffee powder and provide the identified water supply amount, etc. to the refrigerator 1 or the terminal device 80 having the application installed for the refrigerator 1.

The identified water supply amount may be provided to a user through the display 62 of the refrigerator 1 or the display of the terminal device 80. Here, a plurality of water supply amounts may be provided, and a user may select a water supply amount based on a user′ tastes, such as light, regular, strong, etc., among the plurality of provided water supply amounts

According to an embodiment, the processor 67 may control the water supplying unit 65 to adjust the amount of control water supplied to the storage 21 in response to the water supply amount identified corresponding to the kind of coffee powder. For example, when it is identified that 200 ml of cold water is required to prepare a regular concentration of coffee with the coffee powder of a brand A, a user may not separately select the water supply amount or concentration in the cold brew mode. In this case, the processor 67 controls the water supplying unit 65 to supply water by adjusting the water supply amount into 200 ml.

In the foregoing refrigerator according to an embodiment of the disclosure, various beverages such as cold brew coffee are prepared through the water supplying function of the refrigerator 1.

Further, a user may select the water purification mode and the beverage mode as necessary, and select the water supply amount or the concentration in the beverage mode, so that the beverage can be prepared according to the user's tastes.

Further, it is more convenient for a user because the guidance on the preparation of the beverage is provided.

Although a few exemplary embodiments of the disclosure have been described in detail, various changes may be made in these exemplary embodiments without departing from the scope defined in the appended claims.

What is claimed is:

1. A refrigerator comprising:
a main body including a storage compartment;
a door to open and close the storage compartment; and
a beverage provider arrangeable with respect to the main body or the door,
the beverage provider comprising:
a water supplying unit;
a storage to store water supplied by the water supplying unit;
a filter, arrangeable inside the storage, to accommodate beverage powder so that while the filter is arranged inside the storage, ingredients from the beverage powder accommodated in filter are leached into the water stored in the storage;
a sensor to obtain information related to a concentration of a beverage produced to contain the ingredients of the beverage powder in the water; and
a processor configured to identify the concentration of the beverage based on the obtained information, and control the water supplying unit to supply the water in an amount which is adjusted based on the identified concentration.

2. The refrigerator of claim 1, wherein the sensor is among a sensor unit which includes a contact or non-contact concentration sensor to detect the concentration of the beverage based on electrical conductivity.

3. The refrigerator of claim 1, wherein
the sensor is among a sensor unit which includes a weight sensor under the storage, and
the processor is configured to identify weight of the beverage powder accommodated in the filter based on a detection result of the weight sensor, and identify the concentration of the beverage based on the identified weight of the beverage powder and the amount of the water supplied to the storage.

4. The refrigerator of claim 1, wherein
the sensor is among a sensor unit which includes a mounting sensor to detect whether the storage is attached or detected with respect to the beverage provider, and
the processor is configured to count a beverage preparation time from a point in time when the mounting sensor detects that the storage is attached or detected with respect to the beverage provider.

5. The refrigerator of claim 1, wherein the processor is configured to identify the concentration of the beverage based on a preparation time of the beverage exceeding a predetermined reference period of time.

6. The refrigerator of claim 5, wherein the processor is configured to control the water supplying unit to supply the water to the storage, based on the concentration of the beverage identified being higher than a concentration set by a user.

7. The refrigerator of claim 5, wherein the processor is configured to output a message informing that preparation of the beverage is complete, based on the concentration of the beverage identified to reach a concentration set by a user.

8. The refrigerator of claim 5, wherein the processor is configured to output a message that an additional leaching time is required, based on the concentration of the beverage identified being lower than a concentration set by a user.

9. The refrigerator of claim 1, further comprising:
an input unit to receive an input of a user,
wherein the processor is configured to control the water supplying unit to supply the water in the amount which is adjusted based on a water supply amount or concentration of the beverage that is input through the input unit.

10. The refrigerator of claim 1, further comprising an input unit to allow a selection to operate the beverage provider in either a water purification mode to provide purified water or a beverage mode to provide the beverage.

11. The refrigerator of claim 10, wherein
the processor is configured to:
identify a water level of the water in the storage through the sensor in the water purification mode, and
control the water supplying unit to maintain the water in the storage at a predetermined water level based on a result of the water level identified.

12. The refrigerator of claim 10, wherein
the processor is configured to:
identify a water level of the water in the storage based on an input of a user to switch operation of the beverage provider from the water purification mode to the beverage mode, and
perform control not to switch to the beverage mode based on the water level being identified as a predetermined level or higher.

13. The refrigerator of claim 10, wherein
the input unit further receives an input of a user to select a water supply amount or concentration of a beverage in the beverage mode, and
the processor is configured to control the water supplying unit to supply the water in the amount which is adjusted based on the selected water supply amount or concentration, to the storage.

14. The refrigerator of claim 1, further comprising a communicator to communicate with a server, wherein
the beverage powder comprises coffee powder, and
the processor is configured to identify a kind of the coffee powder which is fed into the filter, receive information about a water supply amount or leaching time corresponding to the kind of the coffee powder identified from the server, and control cold brew coffee to be prepared based on the received information.

15. A method of controlling a refrigerator which includes a beverage provider which is arrangeable with respect to a main body or a door of the refrigerator, the beverage provider including a water supplying unit and a storage to store water supplied by the water supplying unit, the method comprising:
performing filtering to leach ingredients from beverage powder, which is accommodated in a filter into water supplied to the storage, the filter being arrangeable inside the storage of the beverage provider;
obtaining, by a sensor, information related to a concentration of a beverage produced to contain the ingredients of the beverage powder in the water; and
identifying the concentration of the beverage based on the obtained information, and controlling an amount of the water supplied by the water supplying unit to be adjusted based on the identified concentration.
